(19) United States
(12) Patent Application Publication
(10) Pub. No.: US 2026/0199010 A1
(43) Pub. Date: Jul. 16, 2026

(54) CHILLED MIST-BASED COOLING APPARATUS

(71) Applicant: DDC Technologies, Inc., Oceanside, NY (US)

(72) Inventors: Benjamin DONSKOY, Oceanside, NY (US); Dmitry DONSKOY, Oceanside, NY (US); Petr GNATYUK, Rockville Centre, NY (US); Horacio Jose Valdes GUANTI, Panama City (PA)

(21) Appl. No.: 19/023,856

(22) Filed: Jan. 16, 2025

(51) Int. Cl. A61B 18/20; A61B 18/22

(52) U.S. Cl. CPC A61B 18/203; A61B 2018/2253

(57) ABSTRACT

An improved apparatus for tissue cooling during laser-based procedures is provided, which utilizes a combination of chilled pressurized air and atomized water to rapidly lower tissue surface temperatures. The apparatus includes a continuous-operating delivery system for chilled pressurized air and a volume-controlled device for water, allowing the air-to-water ratio to be precisely adjusted to prevent excessive moisture accumulation on the tissue. The water-air mixture formed within a chilled mixing chamber is discharged through a single-outlet nozzle as a fine mist spray, which is directed at the laser-irradiated tissue to enhance cooling efficiency.

### TECHNICAL FIELD OF THE INVENTION

The present invention relates to the field of laser-based medical and cosmetic procedures. More specifically, the present invention relates to an apparatus and method for cooling tissues during laser-based procedures by using a chilled liquid-air mixture.

### BACKGROUND OF THE INVENTION

Laser-based medical and cosmetic procedures have gained significant popularity due to their precision and effectiveness in treating various conditions, including skin resurfacing, hair removal, and tattoo removal. However, these procedures often generate substantial heat in a target tissue, which can lead to pain, discomfort, and potential thermal damage to the skin. To mitigate these adverse effects, effective cooling methods are essential.

Traditional cooling techniques typically involve direct application of cold air, ice packs, or contact cooling devices that may not provide adequate or uniform cooling across the treatment area. In some advanced systems, cryogen-based cooling has been utilized, where cryogenic substances are applied to the skin to achieve rapid temperature reduction. While effective in delivering extreme cooling, cryogen methods can introduce risks, such as frostbite, skin irritation, and complications related to the handling of cryogenic materials. Moreover, these methods can also create excessive moisture accumulation on the skin, hindering the laser's efficacy and leading to inconsistent treatment outcomes. The use of cooled air or mist in combination with laser procedures has been explored in prior art, but challenges remain in achieving optimal temperature reduction without excessive moisture buildup. Existing systems often lack the capability for continuous operation, leading to delays and inefficiencies during treatments. Furthermore, conventional designs may fail to ensure a uniform distribution of the cooling effect across the treatment area, resulting in suboptimal outcomes.

There remains a need for an improved cooling apparatus that combines chilled air and atomized water to provide effective and rapid cooling of the skin surface while maintaining a controlled moisture environment.

The present invention addresses these challenges by providing a cooling apparatus that continuously delivers a finely atomized mist of chilled liquid and pressurized air directly onto the treatment area. By optimizing a liquid-to-air ratio and employing a unique mixing chamber design, the apparatus enables rapid evaporation and effective cooling, significantly reducing the likelihood of thermal damage to the epidermis. This innovation offers a novel approach to tissue cooling during laser treatments, enhancing patient comfort and treatment efficacy.

### SUMMARY OF THE INVENTION

The present invention provides an improved cooling apparatus that combines chilled pressurized air and atomized water to rapidly and effectively lower skin surface temperatures. The apparatus utilizes a continuous air pumping means and a liquid coolant (e.g., water) volume controlling device to continuously deliver a chilled pressured liquid-air mixture directly to the epidermis during laser-based procedures.

Continuous operation is critical as it allows a laser to operate at high pulse repetition rates, thereby facilitating rapid procedures. Controlling the ratio of chilled pressurized air to atomized liquid (e.g., water) is essential to minimize excessive moisture accumulation on the skin. An appropriate liquid-to-air ratio also induces effective evaporation of the liquid coolant from the skin surface. This evaporation process results in a rapid reduction in the surface temperature of the epidermis.

Another aspect of the present invention is a spray nozzle of the cooling apparatus which comprises a single outlet configured to emit a combined liquid and air stream.

Consequently, a mist spray is produced by utilizing a single outlet for both the liquid coolant and air. The spray is generated within a mixing chamber and subsequently delivered to the nozzle. As a result of the collision between the air jet and liquid within the mixing chamber, the liquid is atomized into fine droplets, forming a fine mist spray.

The liquid-to-air ratio is critical for achieving a desired liquid content within the spray. The liquid-to-air ratio in the spray is regulated by adjusting the liquid flow.

As the air and liquid are mixed within the chilled mixing chamber and discharged through the common nozzle, the liquid is expelled in the same direction as the air. This is significant, as the atomized liquid spray is consistently delivered to the epidermis at the site of the laser-based procedure, thereby reducing the likelihood of tissue damage.

In various implementations, the air pressure within the delivery means of the apparatus is maintained within a range of 0.5 to 6 bar, with a preferred range of 1 to 3 bar.

This specific air pressure range is essential for producing a fine atomized liquid spray and for transporting the mixture through the delivery means to the nozzle and then to the surface of the skin.

It is preferred that the liquid flow (i.e., the volume of liquid per unit time) through the volume-controlled device of the apparatus is established within a range of 0.01 to 10 mL/min, with a more preferred range being from 0.05 to 1.8 mL/min.

Utilizing liquid flows within the specified range is advantageous for effectively cooling the epidermal surface area through the rapid evaporation of fine liquid droplets of the atomized water spray. Specifically, this liquid flow ensures that a sufficient number of fine liquid droplets are deposited onto the epidermal surface area, thereby facilitating a cooling effect. Conversely, the chosen liquid flow rate is sufficiently low to prevent the formation of excessive moisture on the skin. The formation of such a liquid layer is undesirable, as it would significantly impede the evaporation rate of the liquid causing less-than effective cooling and lead to an excessive water accumulation on the patient's skin.

Furthermore, the continuous operation of the volume-controlled device can be precisely regulated to achieve a specific liquid flow rate.

Thus, the optimal size (e.g., diameter) for the nozzle outlet ranges from 0.5 mm to 2 mm, with a preferred range of 0.8 mm to 1.3 mm.

In certain variations, the volume-controlled device operates in a range of 1 to 200 rpm, preferably from 15 to 40 rpm.

To achieve the aforementioned low liquid flow rates, a positive displacement fluid pump may be employed. Positive displacement pumps operate by drawing a fluid into a chamber at the pump inlet and moving it towards the outlet for discharge, maintaining a consistent flow rate regardless of the pressure at the inlet. These pumps can be categorized based on the method used to transfer the liquid, either through rotary or oscillating (reciprocating) mechanisms. It is important to note, however, that rotary positive displacement pumps are less complex in design and operation compared to oscillating pumps, making them a more efficient choice for certain applications.

Furthermore, a peristaltic pump, a subclass of rotary positive displacement pumps, is the preferred configuration for the pumping mechanism of the apparatus.

The required size of the atomized water droplets ranges from 1 to 300 microns, with a more preferred range of 20 to 200 microns.

In certain embodiments, the nozzle comprises a single outlet designed to deliver both gas and liquid to a desired location. Owing to the high pressure, the likelihood of buildup and clogging within the nozzle is minimized.

To achieve lower temperatures of the liquid-air mixture, the liquid is preferably injected prior to entering the chilled chamber. As the mixture passes through the chilled chamber, the temperature is reduced to approximately 0° C.

It should be noted that a liquid (like water) possesses a higher heat capacity than air, making it advantageous to mix the liquid and air prior to contacting the skin in order to achieve optimal cooling effects.

In one aspect, the delivery line and the nozzle are integrated into the handpiece of a laser system. This configuration enables the continuous application of the chilled mist spray, generated by the cooling apparatus, directly at or near the treatment area, which is irradiated by the laser pulses emitted from the handpiece of the laser system.

The preferred temperature range of the mixture upon exiting the chilled chamber is from −10° C. to 15° C., with a more preferred range of −5° C. to 5° C.

### BRIEF DESCRIPTION OF DRAWINGS

FIG. 1 shows a schematic diagram of an apparatus for cooling a tissue according to the present invention.

FIG. 2 shows a cross-sectional view of an air/water chilled mixing chamber.

FIG. 3 shows a laser handpiece to which the cooling apparatus according to the present invention is connected.

FIG. 4 shows a comparison of measured skin surface temperatures during laser irradiation using 4 different cooling methods.

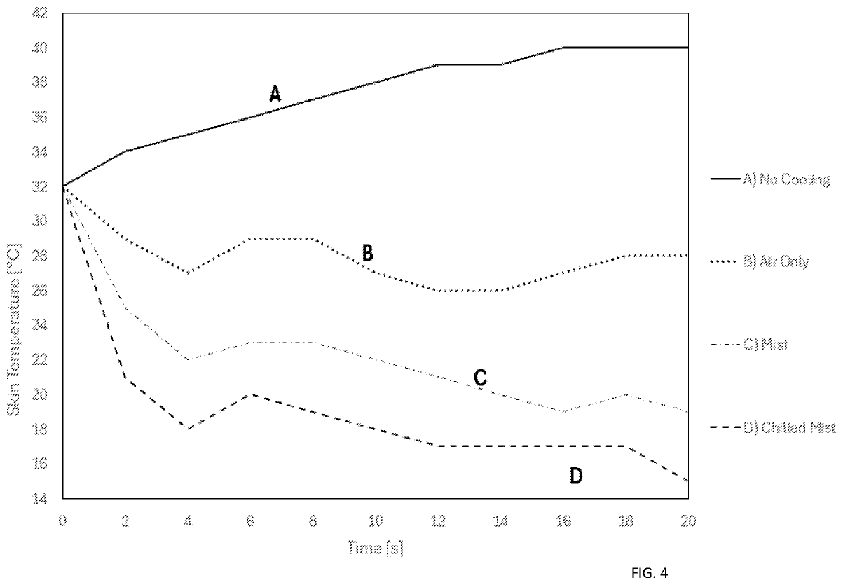

### DESCRIPTION OF THE INVENTION

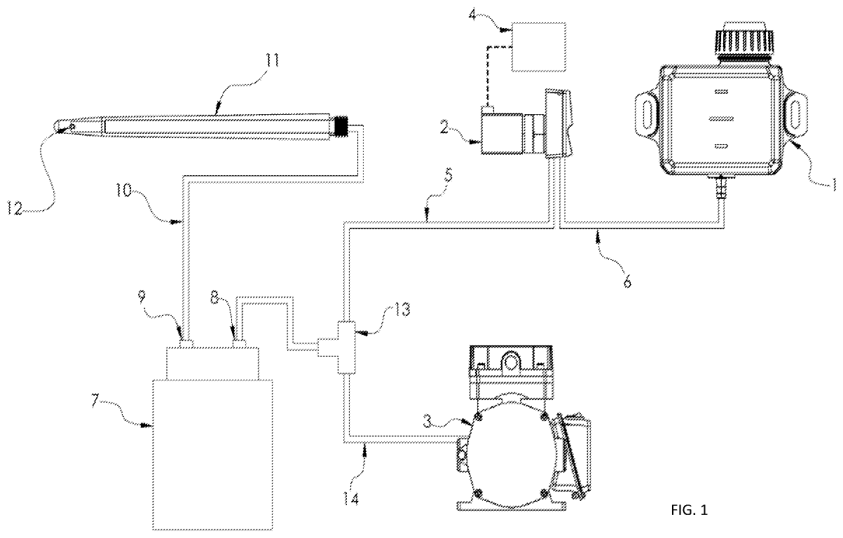

FIG. 1 demonstrates an apparatus for cooling of a tissue, which comprises a fluid reservoir 1, a volume controlling device 2, a gas compressor 3, a chilled chamber 7, a speed controller 4. A single delivery means 10 for a liquid-air mixture is configured to extend to a nozzle 12.

A delivery means 6, in the form of a tube, transfers a liquid coolant from the fluid reservoir 1 to the inlet of the volume controlling device 2, whereas the outlet of the volume controlling device 2 is connected to a three-way junction 13 where it is combined with air supplied by the gas compressor 3. The air is delivered through a tube 14. The liquid coolant flow from the reservoir 1 to the three-way junction 13 is regulated by the volume controlling device 2, wherein the volume controlling device 2 comprises a pump, such as a peristaltic pump, configured to operate in continuous mode with a rotational speed within a range of approximately 1 to 200 revolutions per minute (rpm), and preferably within a range of approximately 15 to 40 rpm.

The air compressor 3 is configured to deliver the air within a constant pressure range of approximately 0.5 to 6 bar, with a preferred pressure range of approximately 1 to 3 bar.

It is noted that, as shown in FIG. 1, the volume controlling device 2 is connected to the speed controller 4, thereby enabling the speed controller 4 to regulate the quantity of the liquid coolant (e.g., water) delivered to the three-way junction 13.

It is further noted that the quantity of the liquid coolant injected into the three-way junction 13 is calibrated to prevent excessive wetting of the target tissue. To ensure optimal delivery, a preferred injection range of approximately 0.01 to 10 mL/min is applied, with a more preferred range of approximately 0.05 to 1.8 mL/min.

As illustrated in FIG. 1, the three-way junction 13 has a delivery tube 5, where the liquid coolant and the air are combined, and is further connected to an input 8 of the chilled chamber 7, which preferably comprises a thermoelectric cooler (TEC) and a heat sink to achieve a substantial temperature reduction. Within the chilled chamber 7, the liquid-air mixture is cooled to subzero temperatures before exiting through an outlet 9.

Furthermore, upon being cooled within the chilled chamber 7, the liquid-air mixture is conveyed through a single tube 10 to the single-outlet nozzle 12 from which it is ejected in a conical spray directly onto the target tissue. Notably, the internal mixing of the liquid coolant and the air within the chilled chamber 7 allows the liquid coolant sufficient time to undergo evaporative cooling. This process results in significant cooling of the surrounding air as the liquid coolant absorbs heat during evaporation. By contrast, external mixing outside the nozzle 12 substantially reduces the contact time between the liquid coolant and the air, thereby decreasing the overall efficiency of heat transfer.

In order to achieve an increased pressure ejection rate, the nozzle 12 is configured with a relatively small orifice, optimally ranging from approximately 0.5 mm to 2 mm in diameter, with a preferred range of about 0.8 mm to 1.3 mm.

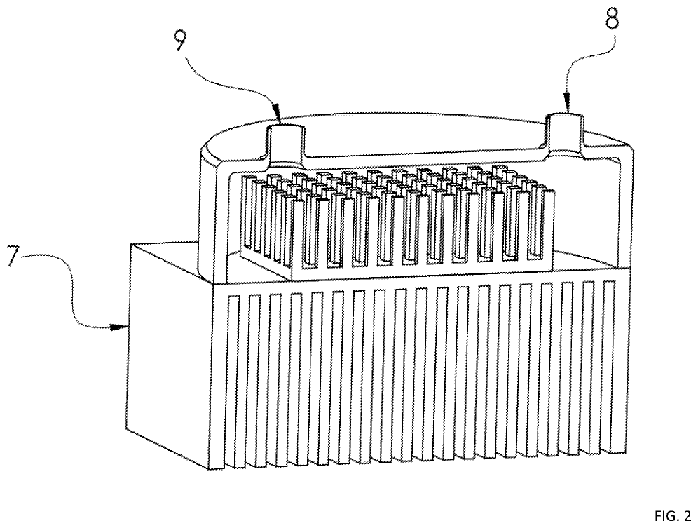

FIG. 2 shows a cross-sectional view of the chilled chamber 7 with its input 8 and output 9. Additionally, a heat sink positioned atop the thermoelectric cooler (TEC) within the chilled chamber 7 is configured to enhance the cooling efficiency of the liquid-air mixture. The rapid flow of the mixture generates turbulent vortices within the chamber, which further enhances the formation of a blended mist mixture, resulting in a finer mist for more uniform cooling upon ejection.

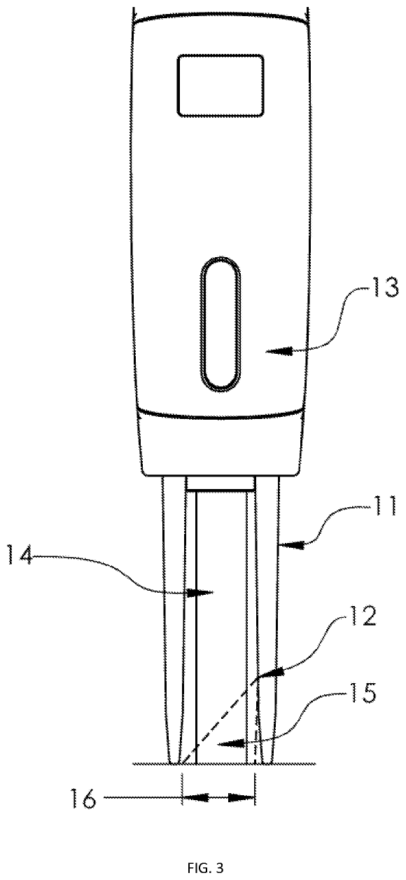

FIG. 3 illustrates an embodiment of a laser handpiece 13 connected to the cooling apparatus, implemented in accordance with the present invention. The laser handpiece 13 is connected to a laser device via a laser beam delivery mechanism, such as an articulated arm or an optical fiber, with the handpiece positioned at the terminal end of the laser beam delivery means, as shown in FIG. 3. Optical components within the handpiece 13 are configured to determine the size and shape of the laser-irradiated area on the target tissue 16.

As illustrated in FIG. 3, a chilled spray 15 is emitted from the nozzle 12, expanding conically as the distance from the nozzle 12 increases. The chilled spray is directed toward the surface of the target tissue 16, with the area of contact on the target tissue 16 being approximately equivalent in size to the maximum spot size of the laser beam 14 emitted from the handpiece 13.

In certain embodiments, a distance gauge may be provided as a spacer positioned between the laser handpiece and the target tissue. This spacing is critical for achieving a desired laser spot size on the tissue. Additionally, it is important that the nozzle 12 is not positioned too close to the target tissue, as insufficient distance would prevent the mixed spray from adequately covering the laser-irradiated area. Conversely, excessive distance from the target tissue is also undesirable, as it may cause the mixed spray to warm during transit through the air, significantly reducing its cooling effectiveness.

Thus, the optimal distance for the nozzle outlet ranges from 3 mm to 30 mm, with a preferred range of 5 mm to 20 mm.

Finally, the cooling apparatus of the present invention, configured to generate a chilled mist spray, may be designed as an independent, stand-alone unit capable of integration with various standalone laser devices or for general tissue cooling applications.

1. An apparatus for cooling a tissue during laser-based procedures, comprising:
a gas compressor configured to supply pressurized air and connected to a mixing chamber,
a fluid reservoir containing a liquid coolant and connected to the mixing chamber via a volume controlling device, the volume controlling device being configured to regulate a flow rate of the liquid coolant from the fluid reservoir,
the mixing chamber configured to form a liquid-air mixture by mixing the pressurized air from the gas compressor with the liquid coolant from the fluid reservoir,
a chilled chamber connected to the mixing chamber and comprising a thermoelectric cooler (TEC) and a heat sink, the chilled chamber being configured to chill the liquid-air mixture,
a single delivery tube connected to the chilled chamber and extending to a nozzle,
the nozzle comprising a single outlet configured to emit a spray of the chilled liquid-air mixture directly onto the tissue.

2. The apparatus of claim 1, wherein the volume controlling device is configured to operate continuously to maintain a steady supply of the liquid coolant to the mixing chamber.

3. The apparatus of claim 1, wherein the volume controlling device comprises a positive displacement pump, preferably a peristaltic pump, configured to ensure consistent flow rates of the liquid coolant, independently of an inlet pressure of the liquid coolant.

4. The apparatus of claim 3, wherein the positive displacement pump is configured to operate at a rotational speed of approximately 1 to 200 revolutions per minute (rpm), preferably 15 to 40 rpm.

5. The apparatus of claim 1, wherein the chilled chamber is configured to maintain the liquid-air mixture at a temperature between −10° C. and 15° C., preferably between −5° C. and 5° C.

6. The apparatus of claim 1, wherein the nozzle is configured to emit the spray of the chilled liquid-air mixture with a liquid-to-air ratio that prevents excessive moisture accumulation on the tissue by promoting rapid evaporation of liquid droplets upon contact with a surface of the tissue.

7. The apparatus of claim 1, wherein the single outlet of the nozzle has a diameter ranging from 0.5 mm to 2 mm, preferably from 0.8 mm to 1.3 mm, for producing an increased pressure ejection rate of the chilled liquid-air mixture.

8. The apparatus of claim 1, further comprising a distance gauge positioned between the nozzle and the tissue, the distance gauge being configured to maintain a specified distance for optimal spray coverage of a laser-irradiated area on the tissue during the laser-based procedures.

9. The apparatus of claim 8, wherein the distance gauge is configured to ensure that the nozzle is not positioned too close to or too far from the tissue, with an optimal distance for the single outlet of the nozzle to a surface of the tissue ranging from 3 mm to 30 mm, preferably from 5 mm to 20 mm, thereby allowing the spray of the chilled liquid-air mixture to cover the laser-irradiated area while preventing significant warming of the spray of the chilled liquid-air mixture in transit.

10. The apparatus of claim 1, wherein the gas compressor is configured to supply the pressurized air within a range of 0.5 to 6 bar, preferably within a range of 1 to 3 bar, to facilitate fine atomization and delivery of the liquid-air mixture.

11. The apparatus of claim 1, wherein the volume controlling device is configured to provide the flow rate of the liquid coolant within a range of 0.01 to 10 mL/min, preferably within a range of 0.05 to 1.8 mL/min, to achieve fine atomization without excessive moisture accumulation.

12. The apparatus of claim 1, wherein the mixing chamber is configured to induce a turbulent flow to cause the nozzle to emit the spray of the liquid-air mixture as a homogenous and fine mist for effective evaporative cooling of the tissue.

13. The apparatus of claim 1, wherein the nozzle is configured to emit the spray of the chilled liquid-air mixture as atomized droplets having a diameter ranging from 1 to 300 microns, preferably from 20 to 200 microns, for effective evaporative cooling of the tissue.

14. A method for cooling a tissue during laser irradiation, comprising:
supplying pressurized air to a mixing chamber,
delivering a controlled amount of a liquid coolant to the mixing chamber, thereby forming a liquid-air mixture,
chilling the liquid-air mixture in a chilled chamber to a temperature within a specified range,
ejecting, through a nozzle having a single outlet, the chilled liquid-air mixture as a conical spray onto the tissue,
wherein the conical spray covers an area of the tissue approximately equivalent to a maximum spot size of a laser beam on the tissue.

15. The method of claim 14, wherein the chilled liquid-air mixture is ejected at a pressure sufficient to achieve a fine mist of atomized droplets without substantial accumulation of liquid on a surface of the tissue.

16. The method of claim 15, wherein a liquid-to-air ratio is controlled to maintain rapid evaporation of the atomized droplets upon contact with the tissue, resulting in efficient evaporative cooling.

17. The method of claim 14, further comprising positioning a distance gauge between the nozzle and the tissue to ensure optimal spacing for effective spray coverage and cooling of the area of the tissue.
